(19) United States
(12) Patent Application Publication
(10) Pub. No.: US 2019/0045791 A1
(43) Pub. Date: Feb. 14, 2019

(54) SOLUTION TO PREVENT THE SPREAD OF CLOSTRIDIUM DIFFICILE IN A HEALTH CARE FACILITY

(71) Applicant: Parasol Medical LLC, Buffalo Grove, IL (US)

(72) Inventor: Patrick E. Eddy, Allendale, MI (US)

(21) Appl. No.: 16/043,827

(22) Filed: Jul. 24, 2018

Related U.S. Application Data
(60) Provisional application No. 62/543,684, filed on Aug. 10, 2017.

(51) Int. Cl. A01N 59/00; A01N 55/00; A01N 37/16; A61L 2/18

(52) U.S. Cl. CPC A01N 59/00; A61L 2/186; A01N 37/16; A01N 55/00

(57) ABSTRACT

A solution to prevent the spread of Clostridium difficile in a health care facility comprises 3-(trihydroxysilyl) propyldimethyloctadecyl ammonium chloride and either peracetic acid or hydrogen peroxide, or a combination of both peracetic acid and hydrogen peroxide. The 3-(trihydroxysilyl) propyldimethyloctadecyl ammonium chloride can be between 0.10 percent and 10 percent by weight of the solution. The peracetic acid can be between 0.10 percent and 10 percent by weight of the solution. The hydrogen peroxide can be between 1 percent and 10 percent by weight of the solution. A method of preventing the spread of Clostridium difficile in a health care facility comprises entering a room in said facility that has a surface where Clostridium difficile is present and applying said solution.

### CROSS-REFERENCE TO RELATED APPLICATION

This patent application claims priority under 35 U.S.C. § 119(e) upon U.S. Provisional Patent Application No. 62/543,684, entitled “SOLUTION TO PREVENT THE SPREAD OF CLOSTRIDIUM DIFFICILE IN A HEALTH CARE FACILITY,” filed on Aug. 10, 2017, the entire disclosure of which is incorporated herein by reference.

### BACKGROUND OF THE INVENTION

The bacterium Clostridium difficile continues to pose problems for health care facilities. Clostridium difficile is sometimes present in the feces of a patient located in a health care facility. A worker in the health care facility may then touch the feces and thereafter spread the Clostridium difficile to surfaces in other rooms in the facility. The Clostridium difficile may then spread to another patient in the facility, causing illness. The illness can be fatal.

### SUMMARY OF THE INVENTION

According to one aspect of the present invention, a solution to prevent the spread of Clostridium difficile in a health care facility comprises 3-(trihydroxysilyl) propyldimethyloctadecyl ammonium chloride, and either peracetic acid (also known as peroxyacetic acid) or hydrogen peroxide, or a combination of both peracetic acid and hydrogen peroxide. The 3-(trihydroxysilyl) propyldimethyloctadecyl ammonium chloride can be between 0.10 percent and 10 percent by weight of the solution. The peracetic acid can be between 0.10 percent and 10 percent by weight of the solution. The hydrogen peroxide can be between 0.1 percent and 10 percent by weight of the solution.

According to another aspect of the present invention, a method of preventing the spread of Clostridium difficile in a health care facility comprises entering a room in a health care facility that has a surface where Clostridium difficile is present, presenting the above described solution comprising 3-(trihydroxysilyl) propyldimethyloctadecyl, peracetic acid, ammonium chloride and hydrogen peroxide, and applying the solution to a surface in the room. The health care facility can be a nursing home. The health care facility can be a hospital.

According to yet another aspect of the present invention, a method of preventing the spread of Clostridium difficile in a health care facility comprises entering a room of a health care facility where a soiled diaper was exposed, presenting the above-described solution comprising 3-(trihydroxysilyl) propyldimethyloctadecyl ammonium chloride and either peracetic acid or hydrogen peroxide, or a combination of both peracetic acid and hydrogen peroxide, and applying the solution to a surface in the room. The health care facility can be a nursing home. The health care facility can be a hospital.

These and other features, advantages, and objects of the present invention will be further understood and appreciated by those skilled in the art by reference to the following specification, claims, and appended drawings.

### BRIEF DESCRIPTION OF THE DRAWINGS

In the drawings:

FIG. 1 is a flow sheet for a method to prevent the spread of Clostridium difficile in a health care facility; and

FIG. 2 is a flow sheet for another method to prevent the spread of Clostridium difficile in a health care facility.

### DETAILED DESCRIPTION

It is to be understood that the specific devices and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

A novel solution to prevent the spread of Clostridium difficile in a health care facility is herein presented. The solution includes 3-(trihydroxysilyl) propyldimethyloctadecyl ammonium chloride and either peracetic acid or hydrogen peroxide or a combination of both peracetic acid and hydrogen peroxide, and other inactive ingredients. The 3-(trihydroxysilyl) propyldimethyloctadecyl ammonium chloride can be present in an amount between 0.10 percent and 10 percent by weight of the solution. The peracetic acid, if present, can be present in an amount between 0.10 percent and 10 percent by weight of the solution. The hydrogen peroxide, if present, can be present in an amount between 0.10 percent and 10 percent by weight of the solution. For example, the solution can include about 1 percent by weight 3-(trihydroxysilyl) propyldimethyloctadecyl ammonium chloride, about 0.14 percent by weight peracetic acid, and about 0.64 percent by weight hydrogen peroxide. Such solutions provide both quick elimination of Clostridium difficile present on a surface and long-lasting ability to destroy Clostridium difficile that may contaminate the surface in the future.

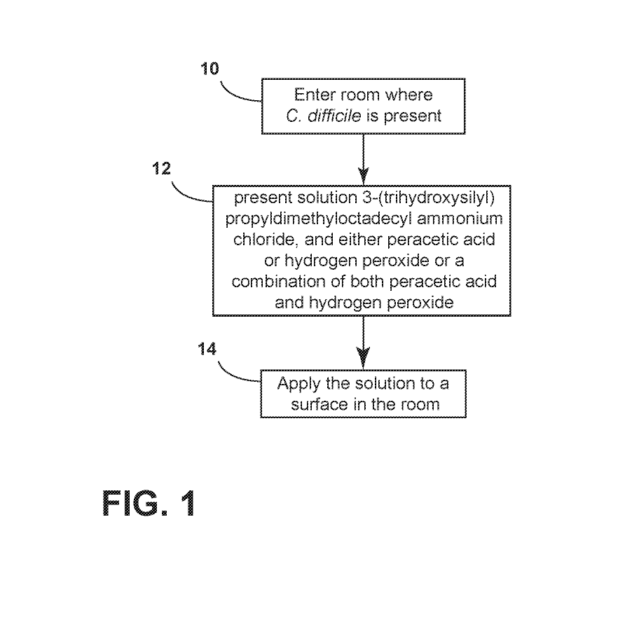

Referring to FIG. 1, a novel method of preventing the spread of Clostridium difficile in a health care facility utilizing the above-described solution is herein presented. The method includes entering a room in a health care facility comprising a surface where Clostridium difficile is present (10), presenting a solution comprising 3-(trihydroxysilyl) propyldimethyloctadecyl ammonium chloride, and either peracetic acid or hydrogen peroxide or a combination of both peracetic acid and hydrogen peroxide (12), and applying the solution to a surface in the room (14). As an alternative, the method may include entering a room in a health care facility comprising a surface where Clostridium difficile is not known to be present but it is desired to prevent Clostridium difficile from contaminating the surface in the future. In such a circumstance, the method again further includes presenting a solution comprising 3-(trihydroxysilyl) propyldimethyloctadecyl ammonium chloride and either peracetic acid or hydrogen peroxide or a combination of both peracetic acid and hydrogen peroxide, and applying the solution to a surface in the room (14). A “health care facility” includes, but is not limited to, facilities such as a nursing home, a hospital, and any facility where a patient is housed or treated.

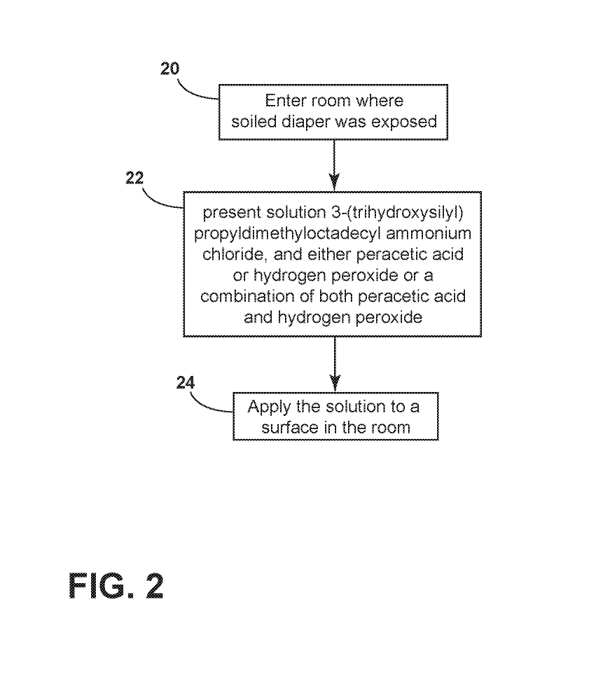

Referring to FIG. 2, another method of preventing the spread of Clostridium difficile in a health care facility utilizing the above-described solution is herein presented. The method includes entering a room of a health care facility where a soiled diaper was exposed (20), presenting a solution comprising 3-(trihydroxysilyl) propyldimethyloctadecyl ammonium chloride and peracetic acid or hydrogen peroxide or a combination of both peracetic acid and hydrogen peroxide (22), and applying the solution to a surface in the room (24). Applying the solution to the surface will destroy Clostridium difficile that transferred from the feces to the surface and will prevent Clostridium difficile from contaminating the surface in the future.

The solution may be applied to a surface in the room via spraying the solution onto the surface. Alternatively, a wipe may be wetted with the solution and the solution applied to the surface via wiping the wipe containing the solution onto the surface.

Surfaces of particular importance to prevent the spread of Clostridium difficile include bathroom surfaces, surfaces where a diaper was changed, surfaces in contact with feces, patient beds, and diaper boxes. The solution can be applied to such surfaces.

The invention claimed is:

1. A solution to prevent the spread of Clostridium difficile in a health care facility comprising:
3-(trihydroxysilyl) propyldimethyloctadecyl ammonium chloride; and
either peracetic acid or hydrogen peroxide, or a combination of both peracetic acid and hydrogen peroxide.

2. The solution to prevent the spread of Clostridium difficile in a health care facility of claim 1,
wherein, the 3-(trihydroxysilyl) propyldimethyloctadecyl ammonium chloride is between 0.10 percent and 10 percent by weight of the solution.

3. The solution to prevent the spread of Clostridium difficile in a health care facility of claim 2,
wherein, the peracetic acid is between 0.10 percent and 10 percent by weight of the solution.

4. The solution to prevent the spread of Clostridium difficile in a health care facility of claim 3,
wherein, the hydrogen peroxide is between 0.10 percent and 10 percent by weight of the solution.

5. The solution to prevent the spread of Clostridium difficile in a health care facility of claim 4,
wherein, the 3-(trihydroxysilyl) propyldimethyloctadecyl ammonium chloride is about 1 percent by weight of the solution.

6. The solution to prevent the spread of Clostridium difficile in a health care facility of claim 5,
wherein, the peracetic acid is about 0.14 percent by weight of the solution.

7. The solution to prevent the spread of Clostridium difficile in a health care facility of claim 6,
wherein the hydrogen peroxide is about 0.64 percent by weight of the solution.

8. A method of preventing the spread of Clostridium difficile in a health care facility comprising:
entering a room in a health care facility comprising a surface where Clostridium difficile is present;
presenting a solution comprising:
3-(trihydroxysilyl) propyldimethyloctadecyl ammonium chloride; and
either peracetic acid or hydrogen peroxide or a combination of both peracetic acid and hydrogen peroxide; and
applying the solution to a surface in the room.

9. The method of claim 8, wherein, the 3-(trihydroxysilyl) propyldimethyloctadecyl ammonium chloride is between 0.10 percent and 10 percent by weight of the solution.

10. The method of claim 9, wherein, the peracetic acid is between 0.10 percent and 10 percent by weight of the solution.

11. The method of claim 10, wherein, the hydrogen peroxide is between 0.10 percent and 10 percent by weight of the solution.

12. The method of claim 11, wherein the health care facility is a nursing home.

13. The method of claim 11, wherein the health care facility is a hospital.

14. A method of preventing the spread of Clostridium difficile in a health care facility comprising:
entering a room of a health care facility where a soiled diaper was exposed;
presenting a solution comprising 3-(trihydroxysilyl) propyldimethyloctadecyl ammonium chloride, peracetic acid, and hydrogen peroxide; and
applying the solution to a surface in the room.

15. The method of claim 14, wherein, the 3-(trihydroxysilyl) propyldimethyloctadecyl ammonium chloride is between 0.10 percent and 10 percent by weight of the solution.

16. The method of claim 15, wherein, the peracetic acid is between 0.10 percent and 10 percent by weight of the solution.

17. The method of claim 16, wherein, the hydrogen peroxide is between 0.10 percent and 10 percent by weight of the solution.

18. The method of claim 14, wherein the health care facility is a nursing home.

19. The method of claim 14, wherein the health care facility is a hospital.

20. The method of claim 17, wherein the health care facility is a nursing home.
